(19) United States
(12) Patent Application Publication
(10) Pub. No.: US 2019/0274755 A1
(43) Pub. Date: Sep. 12, 2019

(54) THERAPEUTIC ABLATION OF TISSUE USING IMPEDANCE MODULATED RADIO FREQUENCY

(71) Applicant: Medtronic, Inc., Minneapolis, MN (US)

(72) Inventors: William J. Smith, Urbana, IL (US); Ricardo Vallejo, Bloomington, IL (US); David Leonardo Cedeno, Normal, IL (US)

(21) Appl. No.: 16/294,667

(22) Filed: Mar. 6, 2019

Related U.S. Application Data
(60) Provisional application No. 62/640,672, filed on Mar. 9, 2018.

(51) Int. Cl. A61B 18/14; A61B 18/12

(52) U.S. Cl. CPC A61B 18/1482; A61B 2018/00577; A61B 18/1206

(57) ABSTRACT

Techniques are disclosed for ablating a tissue mass with a radio-frequency signal. In some examples, the method includes delivering a radio-frequency signal through an impedance modulation element to an introducer to create a lesion on the tissue mass. The method also includes modulating an impedance of the impedance modulation element, determining an overall impedance of the impedance modulation element and the tissue mass, and controlling an amplitude of the radio-frequency signal based on the determined overall impedance. The method further includes controlling a size of the lesion based on the amplitude of the radio-frequency signal.

This application claims the benefit of U.S. Provisional Patent Application No. 62/640,672 (filed Mar. 9, 2018), the entire content being incorporated herein by reference.

### TECHNICAL FIELD

This disclosure generally relates to therapeutic ablation.

### BACKGROUND

Radiofrequency (RF) based ablation has been developed as a therapy that is utilized in interventional pain medicine and neurosurgery for denervation of neural tissues, in interventional cardiology for correcting fibrillation, and oncology to destroy cancerous tumors, while protecting the integrity of neighboring structures. For example, radio waves of certain intensity are delivered via a probe into tissue. The energy deposited generates a current due to ionic flow, which in turn causes ablation at the tissue.

### SUMMARY

This disclosure describes systems, methods, and techniques for controlling the size of a lesion. As described in more detail, one or more of the example systems may manipulate impedance of the system to control the amount of radio-frequency (RF) power that is delivered to the tissue. For example, the example system perform example algorithms to modulate the impedance of the system to control the RF power, thereby controlling the size of the lesion, while maintaining safe ablation target temperature. The modulation of the impedance of the system in conjunction with customization on the systems power delivery algorithm allows for the control of RF power and ablation volume in a manner that address potential limitations of other RF technologies, while not exceeding operational temperatures, safety regulations, or increasing current and voltage to undesirable levels.

The disclosed system can include a RF generator, a probe with sensing elements, an introducer needle or cannula with an active tip, and a grounding electrode, which may be a pad. The RF generator in the example systems can utilize a power delivery algorithm that is customizable and specific to the desired ablation target. In some examples, the example systems may include a high wattage motorized rheostat, herein referred to as the resistive device, the resistive element, the impedance modulation element, or the impedance modulation component, for modulating the impedance utilized by the RF generator algorithm. The impedance modulation component can have any combination of resistive elements, capacitive elements, and/or inductive elements. The impedance modulation component, in some examples, is comprised of a variable capacitor, variable inductor, or any combination of motorized rheostat, variable capacitor, and variable inductor. The example systems may be able to modulate the impedance detected by the RF generator to create the appropriate profile of the RF current output.

In some examples, the example systems include at least one RF probe, and a grounding electrode with a plug and play configuration designed specifically for interfacing with the impedance modulation component. The grounding electrode may connect to the impedance modulation component and have a separate connection from the device to the ground connection of the RF generator apparatus. The connections from the grounding electrode to the impedance modulation component and to the generator ground may all be internalized within the RF generator housing. In some examples, the RF probe may be specific to its introducer needle or cannula gauge size, resulting in a probe for each introducer size. Additionally or alternatively, the RF probe can have one “universal” size that fits within a variety of introducer needles or cannulas that correspond to different outer diameters (gauge size), while all share the same internal diameter, which is large enough to accommodate the RF probe in order to have optimal capacitive coupling. The RF probe may include a sensing logic that is able to communicate with the microcomputer in the impedance-modulation RF system, which allows the device to sense which introducer size is being used and adjust the RF power output accordingly. The RF probe may also include temperature sensing, impedance sensing devices or systems that allow accurate determinations of the tissue temperature and the overall system impedance.

In some examples, the example systems further include a user interface, where a physician is able to select the size of the ablation lesion that is best suited to the target tissue depending on its anatomical location and surrounding structures. As one example, for neuro-ablation therapy, the user interface may comprise a graphical user interface consisting of a touch screen or LCD with associated physical buttons, allowing the physician to select, from a menu, the ablation location, which will define the optimal lesion size according to the anatomical profile of the target area. For tumor ablation, the example systems could utilize an algorithm and modulate the impedance of the system to adjust the RF power to produce a lesion size that is congruent to a tumor size that is designated by the physician via the user interface.

The example systems may be capable of being programmed by a physician and may use such program information in a way specific to its method of use. The techniques can depend on the specific location of the target tissue to be ablated. For instance, ablation of a tumor may require a specific lesion shape and considerable size to destroy the cancerous cells. In contrast, neuro-ablation may require a particular size that is not necessarily the largest possible size available for a given RF probe and introducer. For example, ablation of sensory innervations in the knee near a motor nerve can use a specific positioning of the probe and a particular lesion size that leads to lesioning the proper sensory innervation while sparing the motor nerve. These therapy-specific programs constitute a variety of methods enabled by the use of the disclosed impedance-modulated system. Other uses may exist for other therapies such as cardiac RF ablation or focal lesioning in the brain. The disclosed systems may not only determine and control lesion size and shape but may also suggest the introducer size that provides minimal tissue trauma to the patient.

The ability for site-specific or physician-chosen changes in lesion size and shape through a programmable RF device as described herein provides a new paradigm in RF ablation for therapeutic uses. The example systems can use a set temperature value for therapeutic benefit. The device may be configured to maintain and not exceed the temperature by tracking the temperature using a temperature sensor near the probe-tissue interface. Through modulation of the total system impedance, the RF power may be increased using current or voltage increments and may have less deviation from thermal equilibrium than other systems. The example systems may be configured to custom control lesion sizes and shapes while delivering safe levels of RF current, voltage, and power.

In one example, the disclosure describes a method for ablating a tissue mass, where the method includes delivering a radio-frequency signal through an impedance modulation element to an introducer with an active tip to create a lesion on the tissue mass. The method also includes modulating an impedance of the impedance modulation element, determining an overall impedance of the impedance modulation element and the tissue mass, and controlling an amplitude of the radio-frequency signal based on the determined overall impedance. The method further includes controlling a size of the lesion based on the amplitude of the radio-frequency signal.

In one example, the disclosure describes a system for ablating a tissue mass, where the system includes an RF probe, an introducer needle or cannula with a conductive active tip, an impedance modulation component, a signal generator configured to deliver a radio-frequency signal through the impedance modulation element to the active tip of the introducer in order to create a lesion of a desired size on the tissue mass. The system also includes processing circuitry configured to modulate an impedance of the impedance modulation component and determine an overall impedance of the impedance modulation component and tissue mass. The processing circuitry is further configured to control an amplitude of the radio-frequency signal based on the determined overall impedance and control a size of the lesion based on the amplitude of the radio-frequency signal.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

The various features and advantages of the present disclosure may be more readily understood with reference to the following detailed description taken in conjunction with the accompanying drawings, wherein like reference numerals designate like structural elements.

FIG. 1A illustrates conceptually the connection of the impedance-modulation RF system as a plug and play component of the RF generator apparatus in accordance with the present disclosure.

FIG. 1B is a block diagram illustrating conceptually the connectivity between the processing logic, motorized rheostat, RF Generator, and tissue in accordance with the present disclosure.

FIG. 2A illustrates conceptually a motorized rheostat or potentiometer in accordance with the present disclosure.

FIG. 2B illustrates conceptually a housing for the impedance-modulation RF system with touch screen display in accordance with the present disclosure.

FIG. 3A illustrates the graphical user interface including a touch screen that allows the physician to customize lesion size based on location in accordance with the present disclosure.

FIG. 3B illustrates the graphical user interface, including its ability to reproduce imaging from a magnetic resonance imaging (MRI) device, computerized tomography (CT), or x-ray, to allow for measurement of the necessary lesion size to optimize ablation in accordance with the present disclosure.

FIGS. 4A, 4B, 4C, and 4D are circuit diagrams illustrating the current flow during RF ablation in accordance with the present disclosure.

FIGS. 5A, 5B, and 5C illustrate conceptually flow charts of programming algorithms employed by the impedance-modulation RF system in accordance with the present disclosure.

FIGS. 6A, 6B, 6C, 6D, and 6E illustrate conceptually design of a probe to introducer interface that is optimized for propagation of RF power to the tissues in accordance with the present disclosure.

FIGS. 7A and 7B are a perspective diagram and a cross section view illustrating an example ablation cannula and probe for tissue ablation.

FIG. 8 is a graph of RF lesion size compared to peak power for monopolar RF therapy system in accordance with the present disclosure.

FIG. 9 is a graph of RF lesion size compared to peak power for cooled radiofrequency compared to four groups of ablations performed with a prototype of the system in accordance with the present disclosure.

FIG. 10 is a flowchart illustrating an example operation in accordance with the techniques of the disclosure.

### DETAILED DESCRIPTION

The present disclosure will be more completely understood through the following description, which should be read in conjunction with the drawings. In this description, like numbers refer to similar elements within various embodiments of the present disclosure. The skilled artisan will readily appreciate that the methods, apparatus and systems described herein are merely exemplary and that variations can be made without departing from the spirit and scope of the disclosure.

When radio waves of certain intensity are delivered via a probe into conductive tissue that has adequate impedance, the energy deposited generates a current due to ionic flow. This current flow increases the temperature of the tissue around the probe. A grounding electrode, which may be in the shape of a pad, located away from the radio-frequency (RF) probe and attached to the skin of a patient closes the circuit. When the temperature of the tissue exceeds 45 degrees Celsius, biological changes start to occur in cells. Temperatures above this critical value have an increasingly larger impact. At temperatures above 55 degrees Celsius, an ablation lesion may form. In some contexts, it may be desirable to create a large lesion through manipulation of RF probe configuration, size of probe introducers, ablation time, ablation temperature, and injection of conducting fluids in the lesion site.

Two example paradigms within RF ablation technology are impedance-based control and temperature-based control. Most technologies for tumor ablation aim for large ablation volumes and use an impedance-based control system, wherein the device either presets the power to a certain value or measures tissue impedance and adjusts the RF power applied to the tissue. In both cases, this continues until the detected impedance increases by a set value that indicates charring has occurred, at which point the amount of RF signal applied is changed. Neuro-ablation therapy uses temperature-based control, which focuses on reaching and maintaining a certain temperature requirement. This is accomplished through a temperature-based control algorithm. The outline of such an algorithm is as follows: The temperature of either the tissue surrounding the probe or the probe itself is measured at a given sampling rate. If the temperature measured is less than the desired ablation temperature, usually 80 degrees Celsius, the RF ablation apparatus increases the power delivered to the tissue to be ablated by either increases the current or the voltage delivered to the tissue. This process can continue until the requisite temperature is reached. Usually the time to attain the target temperature is preset and is known as the ramping period. At this point, the system can reduce the applied current or voltage to avoid exceeding 80 degrees Celsius. The RF system then continues to adjust the voltage or current in an effort to keep the measured temperature constant at 80 degrees Celsius and continues this for the user preset duration of the lesioning.

In the case when voltage increment is used to increase the temperature, the resulting RF current delivered to the tissue, which is actually the driver of temperature change in ohmic heating, is a result of Ohm's law: V=iR, where i is the current, V is the voltage, and R is the impedance, usually that of the tissue mass to be ablated. This control method is known herein as voltage-driven mode. The case, in which the RF generator increases the current to increase the temperature, is known herein as current-driven mode. Given the direct relationship between temperature in ohmic heating and current delivered, the current-driven mode can provide for a more direct change in temperature as a change in current is directly proportional to the change in temperature. In the voltage-driven mode, an increase in voltage produces an increase in current proportional to the uncontrollable impedance of the tissue, R, which then produces an increase in temperature. An example system can include algorithms that cause the RF generator to alter between voltage-driven and current-driven modes. More specifically, if the impedance measured by RF system is below a certain preset threshold, the system may increase the temperature using a current-driven mode. Alternatively, if the impedance is above a certain preset value, the system may utilize voltage-driven temperature steps.

As stated above, the principle of RF ablation is ohmic heating, wherein the ablation temperature is the result of thermal motion of electron carrying species in the tissue surrounding the probe as a result of the RF current passed through the tissue. As such, controlling a tissue lesioning process via ablation through temperature may fail to recognize the more direct correlation between lesion size, RF electrical power, and the penetration of the RF signal into tissue. The relationship between lesion size and RF power is well described in the literature. One technique for using power-based control of the lesion size is through the injection of conductive fluid in tissue, which increases the required power, by reducing the impedance of the tissue.

Current or voltage can be modulated in an effort to increase power and therefore lesion size. However, current or voltage modulation alone does not provide full control over lesion size. In neuro-ablation applications, the delicate nature of the surrounding structures makes control of the lesion size and shape critical. Additionally, since the development of the field of neuroablation has focused on large lesions, relatively large diameter introducer needles or catheters are needed to accommodate larger probes, including those that have been developed to cool the RF probe for increased size and distal expansion of the lesion. By utilizing larger introducers, a system can create a larger surface area for RF signal delivery and then induce more electron carrying species into thermal motion. However, some clinicians may prefer to use smaller introducers to, for example, minimize the impact on the surrounding tissue.

An RF ablation apparatus can use high frequency alternating current to take advantage of ohmic heating effects in biological tissues. By sending a high frequency, e.g. typically 300-500 kHz, current through the tissue from an RF probe to ground, the electron carrying species in the tissue surrounding the probe are accelerated in the direction of the resulting electric field. Given that the current is alternating, the electric field is constantly changing direction, resulting in ions and molecules colliding with each other and deviating from their electrically guided movements. This deviation is known as thermal motion and is the process by which electrical energy is converted to thermal energy. Ohmic heating is given that name due to its close relation to Ohm's Law. As such, the heat transfer to the tissue is directly related to system impedance (R), power (P), current (I), and voltage (V) by Equations 1 and 2:

V=I×R Equation 1

P=I²×R=IV Equation 2

In view of these equations and the underlying principles of ohmic heating, the therapeutic ablation size depends on the power delivered. Temperature, on the other hand, is another metric for evaluating efficacy and safety as a result of ohmic heating. However, measurement of temperature at a given temperature sensor near the RF probe-tissue interface provides little information on lesion size and shape. The system of differential equations 3 to 5 show the relationship between heat transfer to a biological tissue and RF heating, which is based on the effect of current and temperature during ohmic heating of a tissue. Wherein the change in temperature over time at a given location

$$( \frac{\partial T}{\partial t} )$$

is dependent on the heat capacity (C), mass density (ρ), thermal conductivity (k), flux density of the temperature gradient (∇²T), the heat source function (Q), and heat loss function (Qₚ). The heat source function (Q) in RF ablation is given by the product of the current density (J) and the intensity of the electric potential field (E). The product of these two properties is equivalent to electrical power density (Watts per cubic meter) The current density (J) and electric field intensity (E) vectors are solved for using the Laplace equation 6, which is governed by the gradient of electrical conductivity (∇σ) and the gradient of electrical potential (∇V). The heat lost function (Qₚ) is estimated by the product of the mass density (ρ), specific heat of blood (c_{bl}), and the temperature difference between the RF location and the blood source (T-T_{bl}).

$$\begin{matrix}ρ C \frac{\partial T}{\partial t} = k {∇}^{2} T + Q - {Q}_{p} & Equation 3 \\ Q = J \cdot E & Equation 4 \\ {Q}_{p} = ρ {c}_{bl} ( T - {T}_{bl} ) & Equation 5 \\ ∇ σ ∇ V = 0 & Equation 6\end{matrix}$$

With respect to equations 1-6, an RF generator that functions through passive power delivery and temperature-based control offers little ability for lesion size and shape optimization. As such, this disclosure describes an RF generator that can provide programmable ablation sizes suitable for a particular need in accordance with the anatomical location of the nerve to be ablated through control of the power delivery algorithm and accounting for the electrical parameters included in equations 1-6.

Two components can be used to optimize and control lesion size during RF ablation. The first component is the optimal control of the RF power delivered to the tissue in order to maximize energy transferred to the tissue. This disclosure describes example techniques to modulate the overall impedance of the system as one way to control the RF power. The modulation of the impedance measured by the RF generator electrically perturbs the system from a steady state condition that sets the delivery of a limited amount of power to maintain a temperature of 80 degrees Celsius. Unlike the impedance-based control systems used in tumor RF ablation applications, the temperature may not exceed 100 degrees Celsius, which could result in the system shutting down as a safety precaution. Instead, and in accordance to this disclosure, the total RF power delivery over the duration of the ablation is modulated (e.g., by modulating the system impedance) as needed by the clinician to obtain the desired lesion size. In other systems, the total system impedance is dependent on the tissue and cannot be modified. These other applications may rely on a passive ablation where the lesion size cannot be controlled by the clinician. These other systems may provide the minimum current and voltage, for a certain tissue impedance, to obtain a large enough RF power to heat and maintain the tissue at 80 degrees Celsius.

In accordance with one or more examples described in this disclosure, a variable system impedance can be used to alter the RF power drawn from the generator while providing and maintaining a feedback loop to bring and maintain the ablation temperature in the neighborhood of 80 degrees Celsius. This creates an active form of RF ablation, where all variables that contribute to RF power are programmable and thus the lesion size is controllable by the clinician.

A second component that can be used to control the lesion size through RF ablation is the ability of current to penetrate into the tissues surrounding the probe and active tip of the introducer needle or cannula. Current penetration is governed by the thermal conductivity (k) and the electrical conductivity (σ), which relate to the ability of the tissues to transmit thermal energy and electrical power. The thermal conductivity of biological tissues can be evaluated both as a solid and as a liquid. The extensive proteoglycan network, which makes up the extracellular matrix, allows heat transfer to occur in a manner similar to a solid lattice, and thus is governed by both free electron flow and lattice vibrations. Yet, the ionic fluid nature of the interstitial and intracellular spaces allows for heat transfer in a manner similar to fluids, where thermal conductivity is a descriptor of molecular collisions. Similarly, electrical conductivity is dependent on the number of electron carrying species in the tissue. As such, the thermal and electrical conductivities at any given point in time are dependent on tissue temperature, tissue perfusion, ion concentration, protein concentration, and electrical charge. By increasing the ability of current (I) to penetrate the tissues, the thermal heat in the form of RF power delivered to those molecules (equation 4) is increased.

During RF ablation, there is an initial ramping period where the amplitude of the radio waves (e.g., RF energy) that is delivered is ramped up (e.g., gradually ramped up in some cases). After the ramping period has ended, the temperature may be held at a constant 80 degrees Celsius, the RF power delivered may be nearly constant, while the perfusion rate, and the ion, and protein concentrations at the probe-tissue interface decrease as the tissue is ablated. The modulation of the impedance of the RF system can alter the power delivery profile of the RF system in order to actively perturb the system from its steady state. In other words, in accordance with one or more example techniques described in this disclosure, the RF delivery system may modify the impedance of the overall system to actively change the amount of energy that is delivered, and therefore allow control over the lesion size that may not available where the system remains in a steady state providing the same amount of power to maintain the same temperature. The active modulation of the impedance of the system induces changes in both RF current and temperature creating a flux of ions and protein carrying fluids. This alters the thermal and electrical conductivities of the surrounding tissue, modulating the propagation of the RF power into the tissue as programmed. Mathematically, changes in the thermal and electrical conductivities induced through impedance modulation can create a new heat transfer profile as reflected in the change in the differential equation system (Equations 3 to 6).

Conceptually, as the modulation of the impedance changes the ability of the RF power to propagate through the tissue, the number of molecules that are accelerated and induced to vibrate or collide is changed, and thus the ability for optimal ohmic heating is induced. Therefore, through the herein described impedance modulation, the system is capable of altering the RF power delivery profile in a customizable manner that allows for optimal biothermal and bioelectrical transfer of energy to the tissues desired for ablation.

The RF ablation algorithms for power delivery are customized according to the desired ablation size or ablation location. Part of the functionality of all these algorithms is complete control over the electrical parameters that are used in the delivery of power to the tissues. Presently, RF systems are capable of both current-driven and voltage-driven control and decide which to implement based on the initial tissue impedance measured by the system. Furthermore, present systems reach a target temperature at the peak of a ramping period, and then maintain a steady state condition that describes an electrically passive ablation. The disclosed RF system described herein include example algorithms that allow for modulation of the system impedance and selection of voltage-driven or current-driven control based on which modality may provide optimal ablation for the desired lesion size in the target tissue, The RF system described in this disclosure is based on recognizing the impedance of the system as an input in the power delivery algorithms, and that its modulation provides an additional locus of control over therapeutic lesion size.

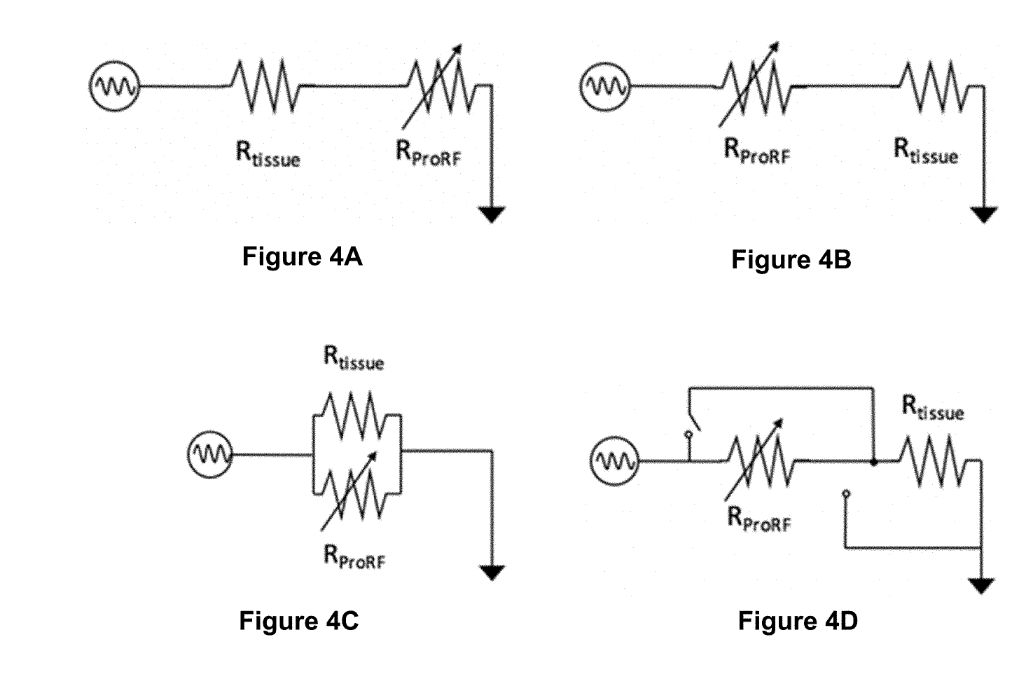

In one example, the impedance modulation is performed through the integration of a programmable, variable, motorized rheostat or potentiometer (e.g., “a resistive device” or “impedance modulation element”), which is connected in series with the biological tissue. This device can be included between the grounding electrode attached to the skin of the patient and the ground input connector of the device, or between the RF output of the device RF and the RF probe. Given that the rheostat or potentiometer is added in series with the tissue as illustrated in FIGS. 4A and 4B, the RF apparatus detects the overall external impedance (R_{Total}) as the sum of the tissue impedance (R_{Tissue}) and potentiometer impedance (Rₚₒₜₑₙₜᵢₒₘₑₜₑᵣ), as shown in Equation 7:

R_{Total}=R_{Tissue}+Rₚₒₜₑₙₜᵢₒₘₑₜₑᵣ Equation 7

The increase in the total impedance (R^{Total}), which is recorded by the RF generator apparatus, can be used to increase the power, by properly using current and voltage to drive the output power. Additionally, altering the external impedance has no direct effect on the tissue itself and thus can be made as high as necessary to reach the desired power. Moreover, as described in more detail, the additional resistance (e.g., R_{Potentiometer}) may be configured to be in parallel with the tissue resistance, which reduces the overall system impedance.

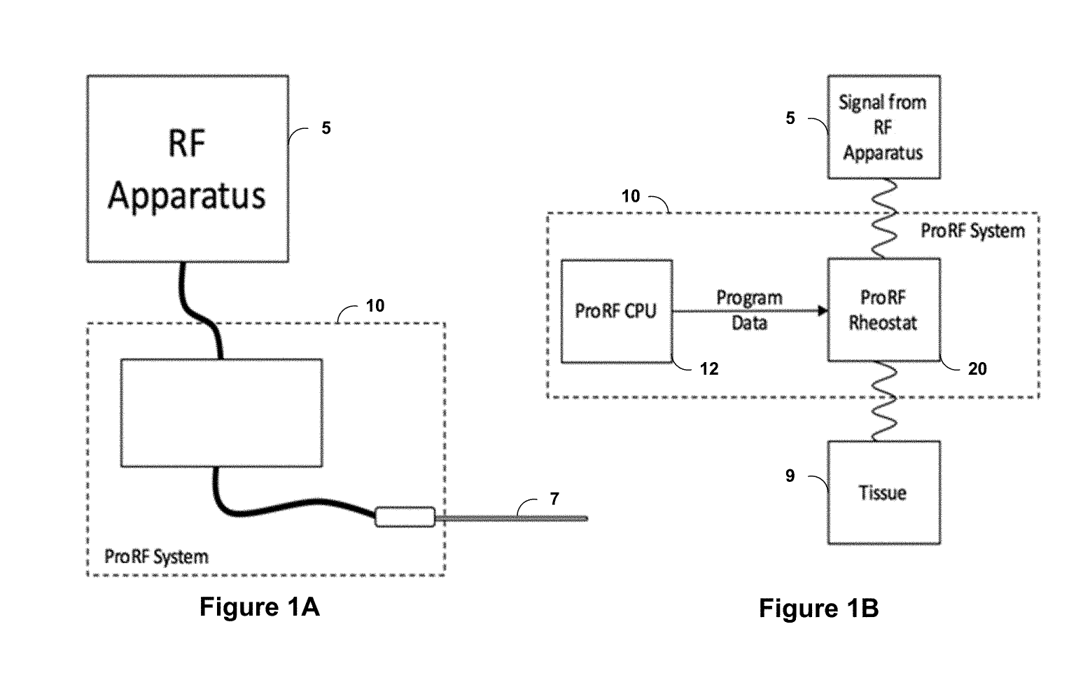

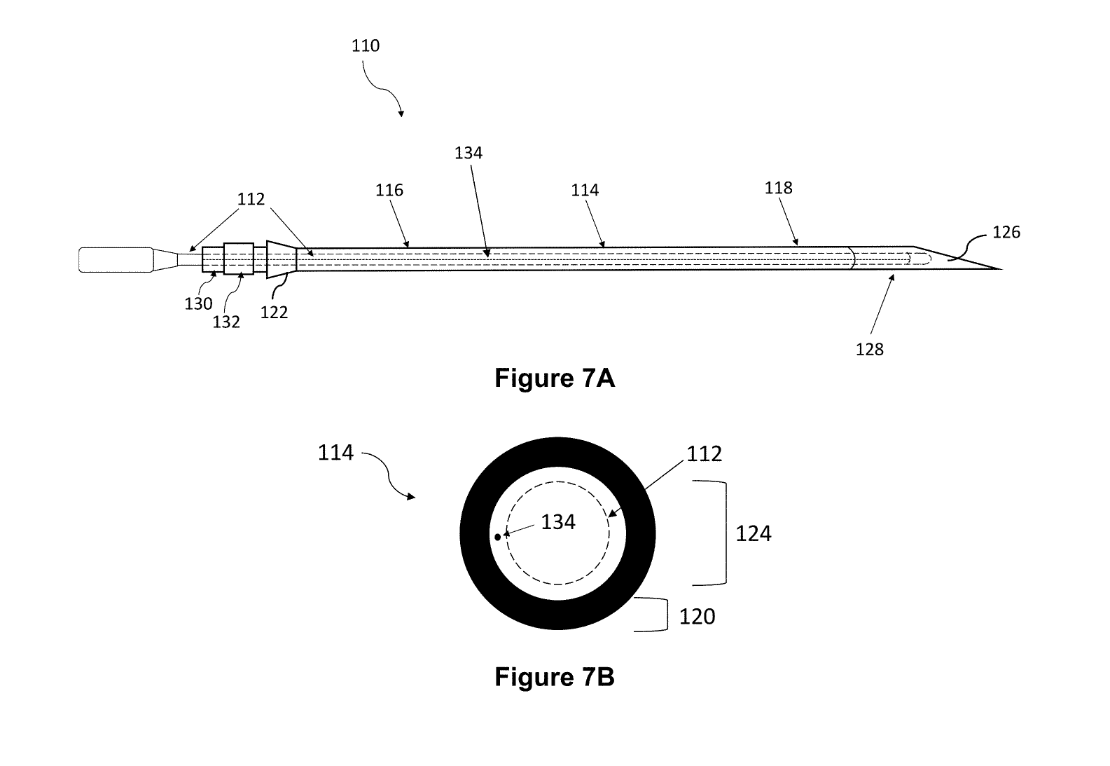

FIG. 1A is a conceptual illustration of the connection of the disclosed impedance-modulation RF device 10 intermediate an RF generator apparatus 5 and a RF probe 7. RF probe 7 can include an introducer needle or catheter with a conductive active tip for delivering an RF signal. Additionally or alternatively, RF probe 7 can include a catheter as shown in FIGS. 7A and 7B. The probe 7 may be attached to the impedance-modulation RF device 10 via a plug and play connection. FIG. 1B is a block diagram illustrating the interconnectivity of the impedance-modulation RF device 10 between an RF generator apparatus 5 and body tissue 9. The impedance-modulation RF device 10 is seen as comprising a processor 12 in communication with a motorized impedance modulation element 20. Processor 12 may be configured to control the impedance of impedance modulation element 20 to modulate the impedance of the RF signal delivered to body tissue 9.

Another system can control the size of the lesion by controlling only the current or voltage generated by a signal generator. However, controlling only the current or voltage does not always provide a sufficient amount of control over the size of the lesion. For example, it may be difficult to create a large lesion size with a small introducer and active tip when controlling only the current or voltage.

In accordance with the techniques of this disclosure, processor 12 can modulate the impedance of impedance modulation element 20 to control the size of a lesion on body tissue 9. Processor 12 can also control the amplitude of an RF signal based on a sensed impedance of the current path. Modulating the impedance and controlling the amplitude provides an additional means of controlling the size of a lesion. Thus, the example systems described herein may be able to create large lesions using small introducer diameters.

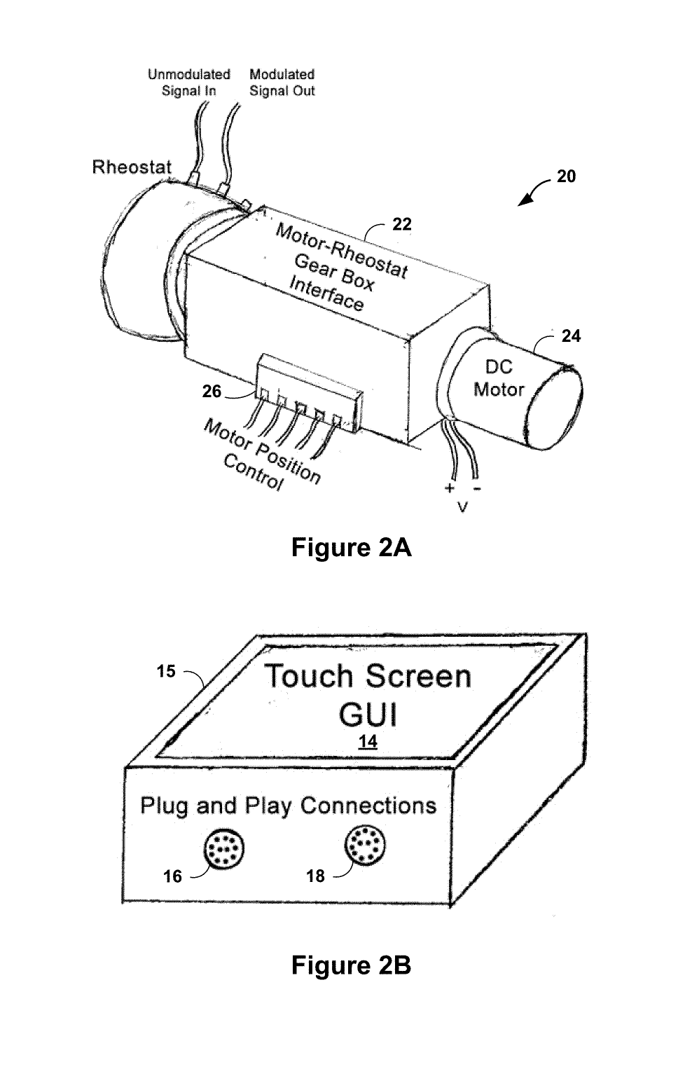

FIG. 2A is a conceptual illustration of a motorized rheostat as comprising an impedance modulation element 20, gear interface 22 and a DC motor 24 capable of having its position specified by a variable voltage or current input. The motorized rheostat shown in FIG. 2A is an example of an impedance modulation element. The processor 12 is capable of controlling the DC motor 24 via motor position control interface 26. Wires connected to the rheostat's variable resistor and wiper, labeled as “Unmodulated signal in” and “Modulated signal out”, are used to modulate the impedance. FIG. 2B is an illustration of the housing 15 enclosing the impedance-modulated RF device 10, a user interface 14 implemented as a touch screen display on the top of housing 15 and two plug and play connector ports 16 and 18, for connection to the RF generator 5 and the probe 7. User interface 14 is an example of an input device for receiving user inputs, such as the selection of a target lesion size, a target amplitude for an RF signal, a defined size of a lesion, a location of body tissue 9, and/or a needle size.

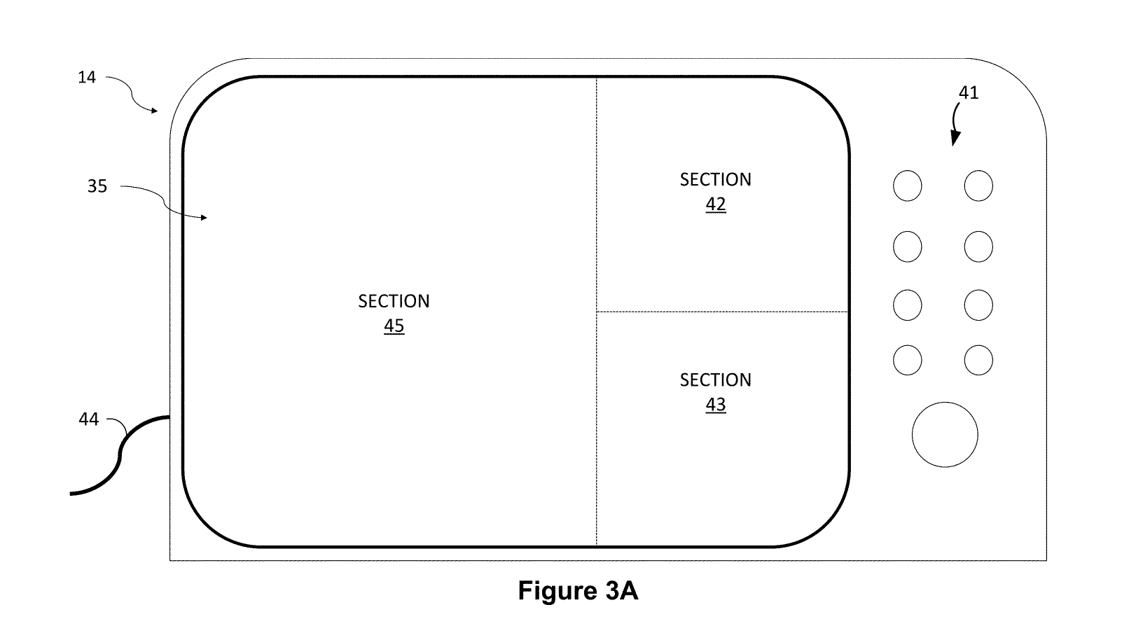

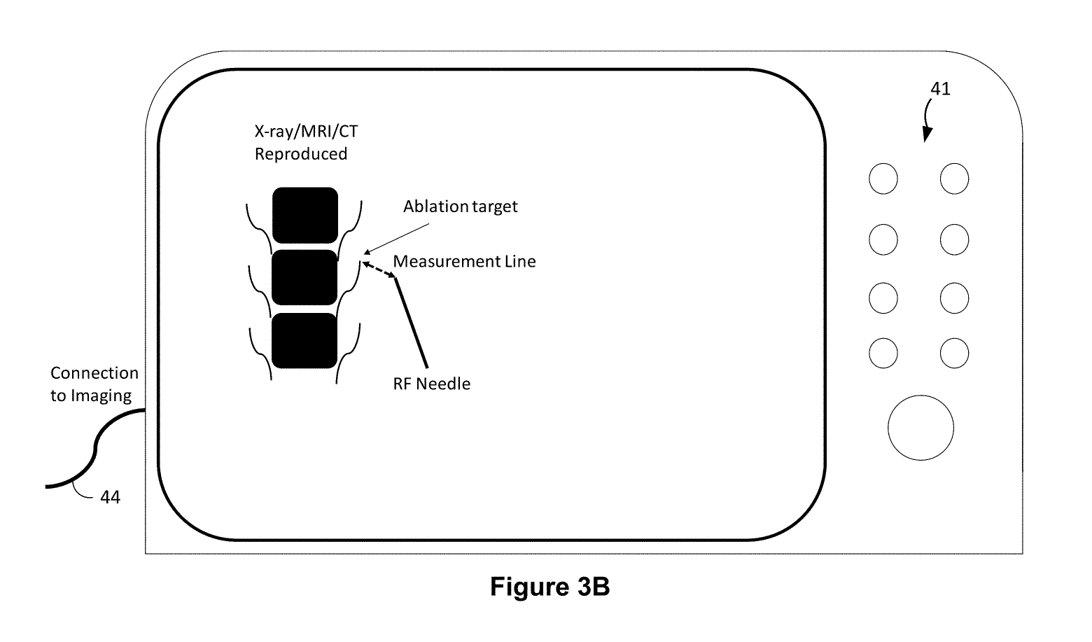

FIG. 3A illustrates the graphical user interface 14 including a touch screen that allows the physician to customize lesion size based on location in accordance with the present disclosure. User interface 14 and visual touch screen interface 35 to allow for communication between the physician and the device. In some configurations, physical buttons 41 can be utilized for altering values on the user interface 14. The touch screen interface 35 is divided into three sections. In some examples, section 42 contains a list of lesion locations used for predetermined lesioning of common anatomical locations. Section 43 contains the custom size entry box is used for physician specification of a certain lesion size outside the recommended programming based on anatomical location allowed by the list of lesion locations 42. An optional connection to the imaging device 44 such as a C-arm or MRI is included in the drawing to allow for Section 45 which contains a reproduction of the imaging allowing the physician to identify the RF probe and lesion margin outline. This can be understood in further detail through FIG. 3B, illustrating in greater detail an example of the RF probe selection and lesion margin drawing interface 35.

FIG. 3B illustrates the graphical user interface, including its ability to reproduce imaging from a magnetic resonance imaging (MRI) device, computerized tomography (CT), or x-ray, to allow for measurement of the necessary lesion size to optimize ablation in accordance with the present disclosure. In the example of FIG. 3B, the physician may be able to specify the exact vertebral location and desired nerve for ablation from a preset list of FDA approved lesion location sites. Additionally, user interface 14 for neuroablation can have an option for programing lesion size directly, through physician entry of the desired volume into the user interface, to accommodate for physician off label use. The physician can select a tumor size based on previous measurements made through medical imaging methodologies such as MRI, CT, or x-ray. The example systems can utilize a user interface that integrates with an imaging output such as x-ray. In this configuration, the physician would then, on the user interface of the system, select the location of the RF ablation probe after placement and designate the location of the nerve or the margin of the ablation target such as a tumor. The system may be configured to calculate the desired parameters needed to create a lesion of that size.

A RF probe 7 suitable for use with device 10 described herein may be implemented with any of radiofrequency compatible probes, including but not limited to cooled probes, multi-tine probes, protruding probes, active fluid injection probes, extended probes, and monopolar, bipolar, or multipolar probe configurations.

In examples described in this disclosure, the motorized impedance modulation element 20 described herein, as referred to as a “impedance modulation element,” may be implemented with a high wattage potentiometer or rheostat with a programmable motor attachment, the motor being capable of receiving voltage, current, or serial input that allows for specified positioning of the potentiometer or rheostat's wiper. In other examples, the motorized impedance modulation element 20 can be exchanged for other passive or active electrical components, including but not limited to capacitors and inductors. In yet another embodiment, impedance modulation may be performed artificially, through altering the impedance input to the power delivery algorithm and inducing a change in the subsequent output values.

FIGS. 4A-4D are representative circuit diagrams illustrating the current flow during RF ablation. The tissue impedance is represented as a fixed resistor labeled R_{Tissue} and the impedance added by the impedance-modulation system is represented by the variable resistor labeled R_{ProRF}. The system impedance (e.g., the “impedance modulation element”) can also include other circuit elements such as capacitors, inductances, switches, diodes, and the like. FIGS. 4A and 4B illustrate circuits in which the impedance-modulation system 10 is placed in series with the tissue. The circuit in FIG. 4A shows the device 10 connected between the grounding electrode in contact with the tissue and the ground connection of the RF generator. The circuit in FIG. 413 shows the impedance-modulation system 10 connected between the RF generator 5 and probe 7, as illustrated in FIGS. 1 and 1B.

The circuit in FIG. 4C shows the impedance-modulation system 10 connected in parallel with the tissue. As compared to a series configuration, the circuit elements of FIG. 4C may experience a higher voltage drop because each circuit element experiences the total voltage drop, rather than only a portion of the total voltage drop. Thus, the voltage drop across each circuit element may increase in FIG. 4C, as compared to a series configuration. FIG. 4D shows a circuit with switches capable of switching the impedance-modulated system 10 between series and parallel configuration. For example, processor 12 can select a first configuration where the impedance-modulation system 10 is connected in parallel with the tissue or a second arrangement where the impedance-modulation system 10 connected in series with the tissue.

In one example, the disclosed impedance-modulation system 10 has a switch that moves the connection such that the motorized impedance modulation element 20 is connected in parallel to the tissue impedance, as shown in FIGS. 4C and 4D. This may be done in situations when the desired lesion size is smaller than the lesion size that would be created based on the natural impedance of the tissue. For example, if the natural impedance of the tissue is very high, a large RF power may be used to reach the lesioning temperature and thus a large ablation lesion will be made. However, if this were to occur in an anatomical region, like the cervical facet joint, the lesion may be too large and impact other structures. By setting the impedance-modulation system to a parallel configuration, the equations governing the overall impedance of the system are:

$$\begin{matrix}{R}_{Total} = \frac{1}{\frac{1}{{R}_{Tissue}} + \frac{1}{{R}_{Potentiometer}}} & Equation 8 \\ or & \\ {R}_{Total} = {R}_{Tissue} ( \frac{x}{x + 1} ) & Equation 9 \\ where & \\ x = \frac{{R}_{Potentiometer}}{{R}_{Tissue}} & Equation 10\end{matrix}$$

The fraction [x/(x+1)] is less than one for any positive value of x, with a limit of 1 as x becomes very large. As such, the total impedance when the impedance modulation element 20 and tissue impedance are in parallel will always be less than that of the tissue impedance on its own. The configurations described are not meant to limit the scope of the disclosure, but rather to provide an example. Any circuit component that interfaces between the RF generator apparatus 5 and the RF probe 7 or the grounding electrode and the RF generator apparatus that alters the overall impedance of the system 10, including capacitors, inductors, and any other passive or active circuit component or components, may be utilized to achieve the results described herein.

The motorized impedance modulation device 20 that is used to modulate power is controlled by a processor 12 that sends a variable output to the position input of the motorized impedance modulation device 20. This positioning is controlled by a voltage or current that varies between zero and some value. In standard industry applications, the positioning is between 0 V and 10 V or 4 mA and 20 mA. These values can be achieved by an analog output from the processor 12 or through a digital potentiometer/rheostat 20 configured as a voltage divider with variable voltage output connected to the motorized potentiometer or motorized rheostat input. The processor 12 can control the motorized rheostat/potentiometer 20 may also be used to control and or power the touch screen panel display of user interface 14 on the exterior of housing 15. The user interface 14 can present a graphic user interface that allows for selection of various programming variable by the user. The information from the user interface 14 may be processed and used to control the motorized potentiometer or motorized rheostat to achieve a specified lesion volume.

In some examples, the components of the impedance modulation RF system include a probe-introducer system designed to enhance penetration of RF power to the tissues through an optimized probe-cannula interface. During RF ablation, the RF current transfers from the electrically active RF probe, through the conductive active tip of the introducer needle or cannula, and finally to the surrounding tissue. Another system may use probes and introducers that leave a relatively large gap due to improper matching of the external diameter of the RF probe and the internal diameter of the introducer. Air or any other non-conductive material that fills the gap acts as a dielectric material, creating a coaxial, concentric capacitor where the RF probe serves as the inner positive plate and the active tip of the introducer serves as the outer plate connected to ground via the tissue. The capacitance (C) of this type of capacitor is described by equation 11. Where L represents the length of the active tip of the introducer, ε₀ is the permittivity of vacuum, which is the ability of an electron to move in a vacuum, Rᵢ is the radius of the inner wall of the introducer and Rₚ is the radius of the RF probe.

$$C = \frac{2 {πɛ}_{0} L}{ln ( \frac{{R}_{i}}{{R}_{p}} )} \qquad Equation 11$$

When the gap between the RF probe and the inner wall of the introducer is filled by air the permittivity is reduced to 1/10059th of its value in vacuum (ε₀). How-ever when the gap is filled with water, the permittivity decreases to ˜1/80th of ε₀, while when the gap is filled with blood, it decreases to ˜1/24000th of ε₀. Thus, the presence of a conductive medium between the capacitor plates (probe and cannula) drives the capacitance of the probe/introducer gap down.

According to Equation 11, the capacitance is related to the relative dimensions of the inner wall of the introducer (R) and the radius of the RF probe (Rₚ). These two determine the length of the gap between the plates of the capacitor, which in this case are the probe and the inner wall of the introducer. When the radii are approximately equal, the gap is almost zero, and the ratio (Rᵢ/Rₚ) is almost one, thus the capacitance tends towards infinity, as the natural logarithm of one is zero. Conversely, as the ratio of the radii increases (i.e. gap increases) the capacitance drops. The capacitance can be reframed in terms of capacitive reactance (X_{C}) as described in equation 12.

$${X}_{C} = \frac{1}{2 π f C} \qquad Equation 12$$

This property quantifies the ability of the capacitor to resist current flow, which is also dependent on the frequency of the RF signal (f). Given the inverse relationship between capacitance (C) and capacitive reactance, a high capacitance means less resistance to current flow. This makes sense intuitively, as to obtain a capacitance of infinity Rᵢ=Rₚ meaning the probe and cannula are in direct contact. Thus a system wherein Rᵢ=Rₚ, represents one where RF power is transferred to the ablation target and thus maximize ablation size.

This optimized probe-introducer interface is embodied by a set of introducers unique to the disclosed RF system. These introducers will be made using biocompatible materials similar to those used in commonly used introducer needles or cannulas. For example, the introducers may have Teflon-insulated shafts and stainless steel active tips, which may vary in length. The outer diameter of the introducers will correspond to the standard diameters of 16 gauge, 18 gauge, 20 gauge, 22 gauge, or any other commonly utilized size. The interior diameter of the introducer will always correspond to one that is large enough to accommodate the diameter of the probe in order to optimize proper capacitive coupling between the probe and the active tip of the introducer. The optimal coupling of the diameter of the RF probe and the internal diameter of the introducer is intended to improve RF current penetration into the ablation target tissue and thus contribute to obtaining reliable lesion sizes. As such, the introducers will have varying wall thicknesses to accommodate the discrepancy between outer diameter (O.D.) and inner diameter (I.D.) This allows for having only one probe with a given size (diameter) to fit all introducers, instead of the commonly used kits consisting of uniquely matched probe and introducer combinations. These can create confusion for physicians in clinic and exacerbate interface issues when incorrectly paired.

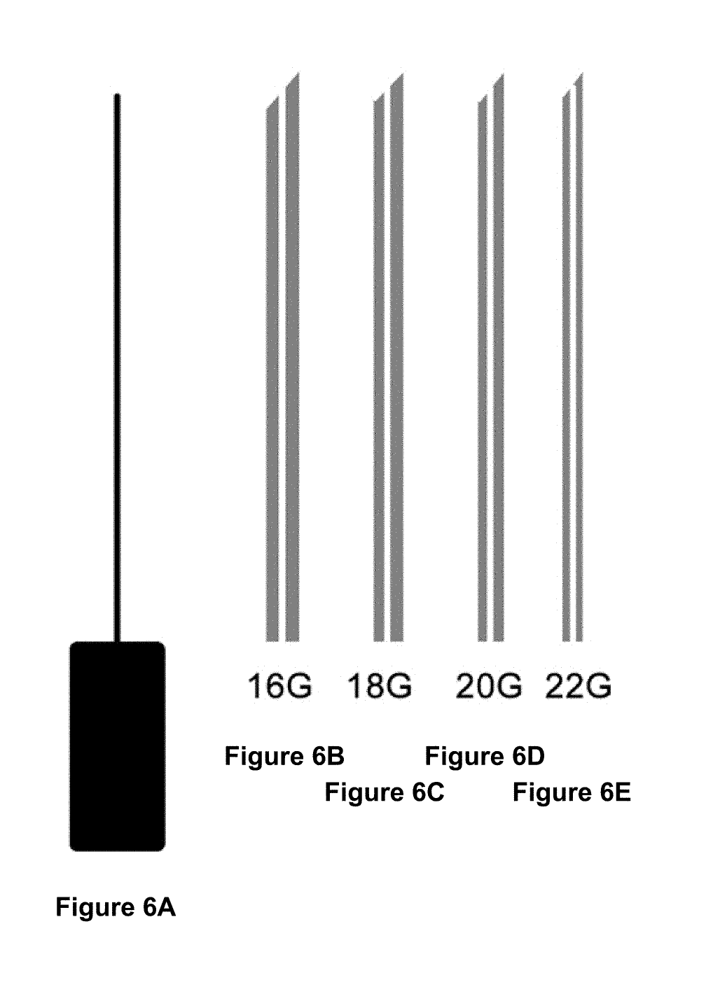

FIGS. 6A-6E are illustrations of the optimized design as disclosed herein. The RF probe 7 has a specific outer diameter size that is constant and suitable for all introducers. The introducers shown correspond to outer diameters equal to that of 16, 18, 20, and 22 gauge and all have a common inner diameter. FIGS. 6B-6E illustrate the varying thickness of the introducer walls. In general, a clinician may prefer a smaller introducer size to limit the impact on surrounding tissue. However, without the techniques of this disclosure, it may be difficult to create a large lesion size with a small introducer. By modulating the impedance of an impedance modulation element, a system of this disclosure provides control of the size of the lesion, as compared to modulating only the current of the RF signal.

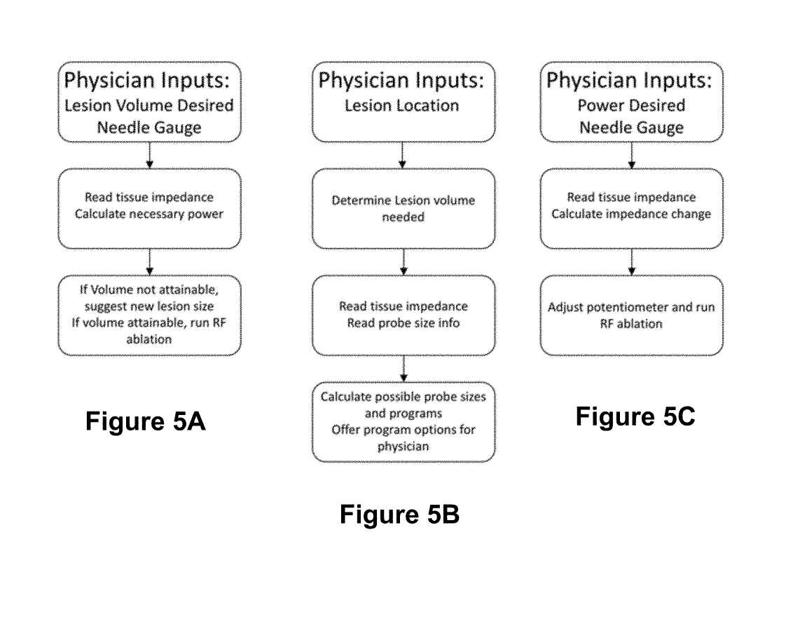

Based on the above physical relations, there is a benefit of algorithm selection and impedance modulation in RF ablation therapies. The algorithm will contain programmable inputs that determine the value of the rheostat or potentiometer, in order to modulate the impedance of the system to any value within an operable range that allows a practical range of the lesion sizes that are suitable to the particular anatomical target. Furthermore, customizable power delivery algorithms can be designed to control various aspects of the ablation lesion such as size and shape. To achieve these customized power delivery algorithms, the impedance modulation system 10 is capable of switching between power delivery profiles based on multitude of inputs and ablation targets. FIGS. 5A-5C are conceptual flow chart illustrations of programming algorithms executable by the processor 12 by the impedance-modulation RF system 10. The data needed to establish this correlation between variables may come from computational modeling and tissue modeling research. These examples represent a selection of possible algorithms but are not intended to limit the possibilities of programming.

To optimize the ability of improved RF power delivery algorithms designed to improved tissue penetration of RF power, the utilization of a probe-introducer interface that is designed to improve RF power delivery is disclosed. Through these mechanisms, the ability to obtain customizable lesions targeted for certain tissues is obtained and may be completed in a shorter time as a result of better thermal energy transfer.

FIG. 5A is a flow chart representing a “Lesion Size-Selection” algorithm, where physicians enter a desired lesion size and introducer size. A physician can enter the desired lesion size by touching a touchscreen display, clicking mouse, or using a keyboard. The “Lesion Size-Entry” algorithm takes in the desired lesion size directly and, in combination with the diameter and length of the active tip of the introducer, calculates the necessary impedances to achieve the power profile and ultimately the desired lesion size. For example, for a larger lesion size and a smaller introducer diameter, processor 12 can initially decrease the impedance of impedance modulation element 20 to increase the initial power delivered to the tissue. When the temperature of the tissue increases to a threshold level, processor 12 may control the current using a control loop based on the temperature and/or impedance of impedance modulation element 20 and the tissue.

FIG. 5B represents a “Location-Selection” algorithm, where the physician selects the location of the target tissue to ablate and the impedance modulation system 10 suggests a program and probe/introducer combination to use. The “Location-Selection” or “Site-Selection” algorithm takes in the desired anatomical target location of the lesion and other factors such as patient size, the algorithm being able to select from this information the adequate size of the lesion needed for therapy and suggest several option for introducer sizes and power profiles able to obtain this lesion size. For example, certain locations in the body can have higher variations in the location of a target tissue mass. For these locations, processor 12 can increase the amplitude of the RF signal to create a larger lesion so that the target tissue mass is ablated.

FIG. 5C is a flow chart of the “Power-Program” algorithm, where in the physicians selects the power to achieve and which program to use. The “Power-Program” algorithm takes in the desired RF power (peak, average, minimum), the desired RF power profile and duration, and executes that power profile. A power profile can be the power levels or amplitude of an RF signal over time. Processor 12 can control the impedance of impedance modulation element 20 based on the selected power profile. The physician can also input the introducer size, and processor 12 can control the impedance of impedance modulation element 20 based on the introducer size and power profile. For example, for a small introducer diameter, processor 12 can decrease the impedance of impedance modulation element 20 to increase the amplitude of the RF signal.

To facilitate communication between the impedance-modulation RF system and other components, a probe designed with a processing logic to allow for communication with the impedance-modulation RF system may be used. The probe may be specific to the introducer size to maximize the amount of contact with the introducer active tip made by the probe. The logic allows for the Impedance-modulation RF system to “sense” the size of the introducer that is being used, allowing for a more seamless user experience as the physician does not have to input the size of the introducer. This aspect may be utilized with the “Site-Selection” algorithm mentioned above, where the size of the introducer and RF probe is dictated to the physician based on lesion location. When a different algorithm is used, where the physician selects the size of the introducer size, the processing logic may generate an output indicating the proper probe size to ensure accurate lesion formation.

Given the wide range and ease of manipulation available using the system described herein, nearly any conceivable power delivery algorithm can be used. Parameters of the power delivery algorithm able to be programmed include initial ramping time and rate, power drop after peak power or temperature equilibrium have been reached, and any manipulations including ramping, step-wise, or gradual changes in power after peak power or temperature equilibrium have been reached. In some examples, the program selection on the device may inform the ramping rate of current and voltage to optimize lesion size and shape. Processor 12 can set the parameters of the power delivery algorithm based on user inputs such as the lesion size, introducer size, lesion location, and/or desired amplitude or power.

FIGS. 7A and 7B are a perspective diagram and a cross section view illustrating an example ablation cannula 110 and probe 112 for ablating tissue. Cannula 110 includes a shaft 114 that has a proximal end 116 and a distal end 118. Cannula 110 is hollow with a thickness 120 and a hub 122 for interfacing with the probe 112 with a diameter 124 that allows for placement within cannula 110. Cannula 110 and probe 112 are examples of the RF probe 7 shown in FIG. 1A.

Distal section 118 includes an ablation electrode 126 located at a distal end 128 of distal section 118. The radio-frequency (RF) energy source, delivers energy to tissue in contact with and proximate to ablation electrode 126 via ablation electrode 126. Other energy sources may be used, such as microwave energy, heat, electrical pulses, ultrasound, cryothermy, and lasers, and the like. As shown in FIG. 7A, the length of ablation electrode 126 in an axial direction relative to distal section 118 may be greater than its width or diameter. For example, ablation electrode 126 may be from 4 to 8 mm long, and 6 or 7 French in diameter.

Cannula 110 includes a handle 130, which in turn includes a manipulator 132. Cannula 110 includes a pull-wire 134 that extends from manipulator 132, through a lumen of cannula 110 that extends through shaft 114 and into distal section 118. By manipulating manipulator 132 to shorten pull-wire 134, an operator may deflect distal section 118 relative to shaft 114 in order to bring ablation electrode 126 into closer contact with target tissue to be ablated. Pull-wire 134 may, as shown in FIG. 7A, be fixed at its distal end to a fixation point (not shown) in order to facilitate the parallel orientation of ablation electrode 126 relative to a target tissue during an application of ablation energy.

Handle 130 may also include an electrical connector (not shown) coupled to an RF energy power source (not shown) and sensing circuitry (not shown). Cannula 110 and Probe 112 may include a variety of other features known in the art, such as a thermistor or thermocouple (not shown) located inside or in thermal contact with ablation electrode 126 to measure the temperature at ablation electrode 126 with thermistor wires (not shown) to couple the thermistor to the connector via a lumen of cannula 110 or probe 112.

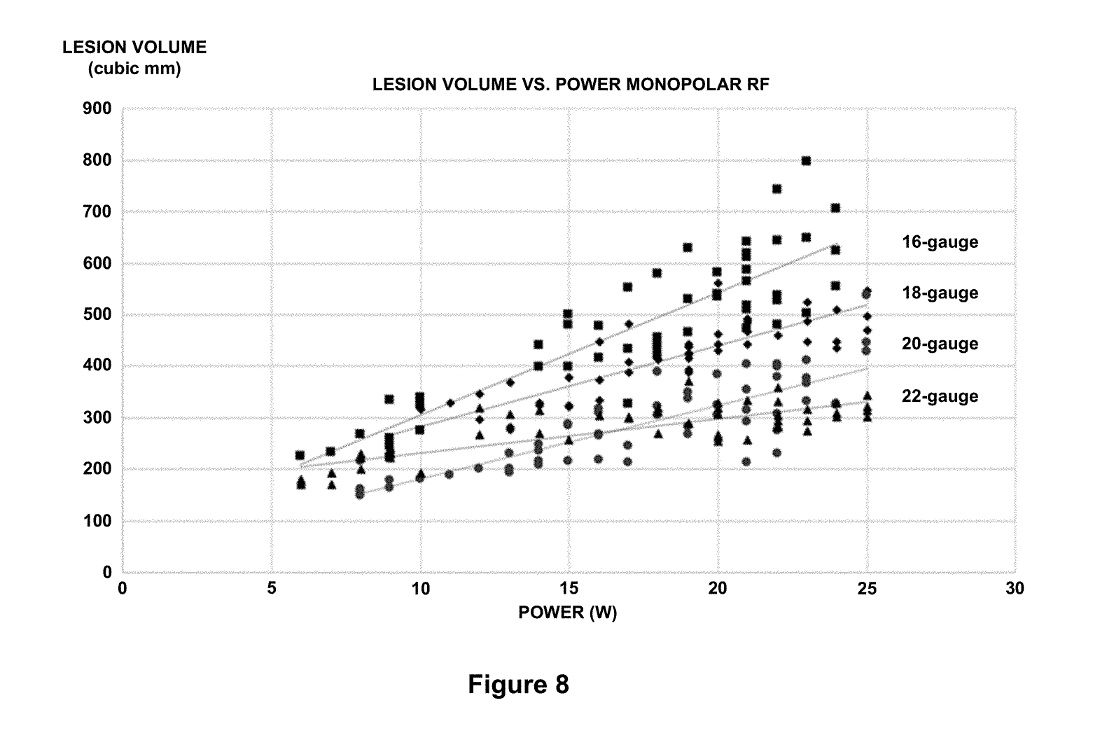

FIG. 8 illustrates a graph in which RF lesion size is correlated to peak output power during monopolar RF therapy. The graphed results were obtained by using a prototype of the system described herein in which the impedance of the system was set at will in order to drive the RF power in proportion to the measured external impedance (R_{Total}), which has been modulated using the potentiometer. FIG. 8 illustrates a strong correlation between peak RF power and the external total impedance, implying that increasing the external impedance detected by the RF device increases the demand for RF power, resulting in larger lesions. The correlation between RF power and the external impedance, (which is the tissue impedance in the absence of the potentiometer) detected by the RF device with lesion size, implies that impedance modulation in order to have control on the output RF power provides a way to control lesion size. Table 1 shows the correlation coefficients for the graphed parameters illustrated in FIG. 8. The trend lines have correlation values between 0.80 and 0.90. While correlation values for the power and starting external total impedance were even stronger, ranging between 0.87 and 0.97.

TABLE 1

| | 16G | 18G | 20G | 22G |
|---|---|---|---|---|
| Peak Power-Initial | R = 0.87 | R = 0.88 | R = 0.88 | R = 0.97 |
| Impedance Correlation | | | | |
| Peak Power-Volume | R = 0.88 | R = 0.91 | R = 0.85 | R = 0.83 |
| Correlation | | | | |

### EXAMPLE 1

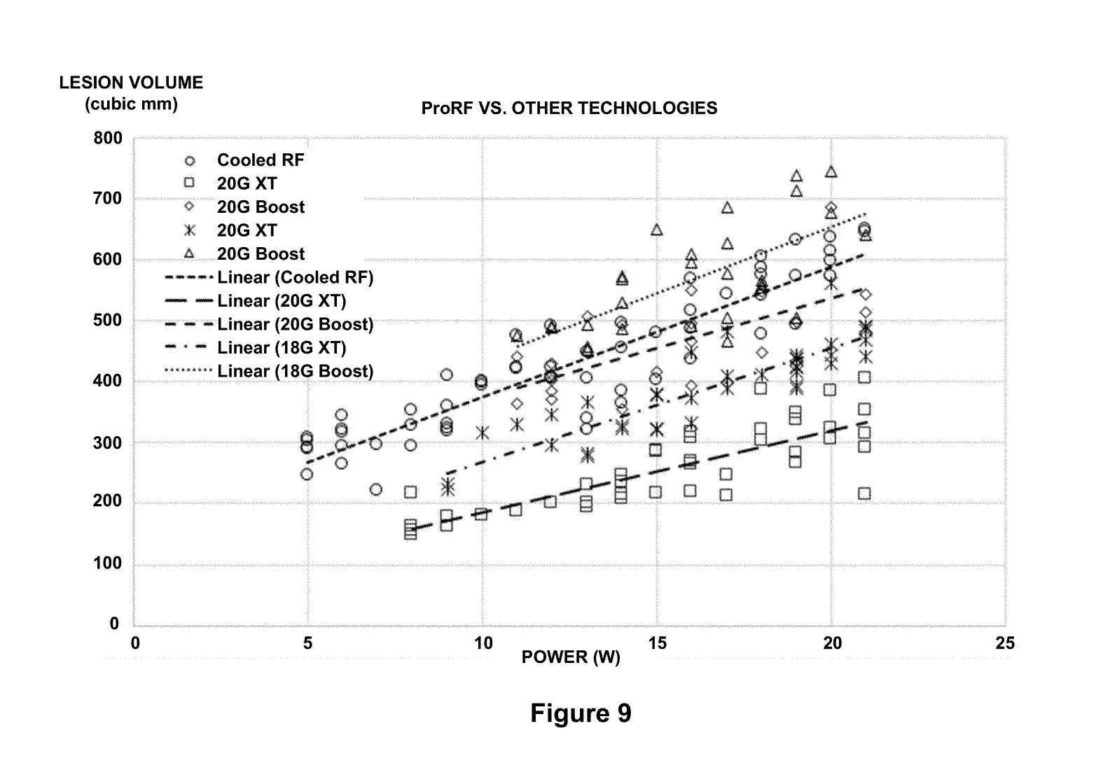

In an in vitro chicken model, a prototype of the described impedance modulation device, comprising a motorized programmable rheostat was connected between a commercially available RF generator grounding electrode and the RF generator's ground. The prototype used two different algorithms, called XT and Boost. In the XT algorithm, the starting impedance was modulated via the motorized rheostat and kept constant for the duration of the ablation. In the Boost algorithm, the impedance was continually varied throughout the ablation using a preset impedance variation pattern with no feedback from the RF device. The results from these ablations were compared against commercially available cooled radiofrequency (CRF). Lesions are modeled as ellipsoids and thus volumes were determined by dissecting the chicken tissue to expose the lesion and then measuring the maximum lengths along the three dimensional axes. Results are illustrated in FIG. 9. CRF, which utilized a 17 G probe-cannula and a 4 mm active tip, produced a range of lesion volumes between 220 mm³ and 650 mm³. The volumes were randomly obtained and depended on the passive algorithm provided by the commercial manufacturer of the RF generator used. The prototype, using an 18 G introducer with a 10 mm active tip, produced lesions in a range of 232 mm³ to 561 mm³ with the XT program depending on the custom selection of the system impedance by the user. The Boost program produced lesions in the range 456 mm³ to 746 mm³ depending on the system impedance selected by the user. Therefore, the prototype provides a total controllable range of lesion volumes between 232 mm³ and 746 mm³ when using an 18 G cannula with a 10 mm active tip. When using a 20 G introducer with a 10 mm active tip, the prototype produced a controllable range of 149 mm³ to 404 mm³ with the XT program, and 356 mm³ to 687 mm³ with the Boost program, giving a total range of 149 mm³ to 687 mm³ for the Prototype and 20 G introducer with a 10 mm active tip.

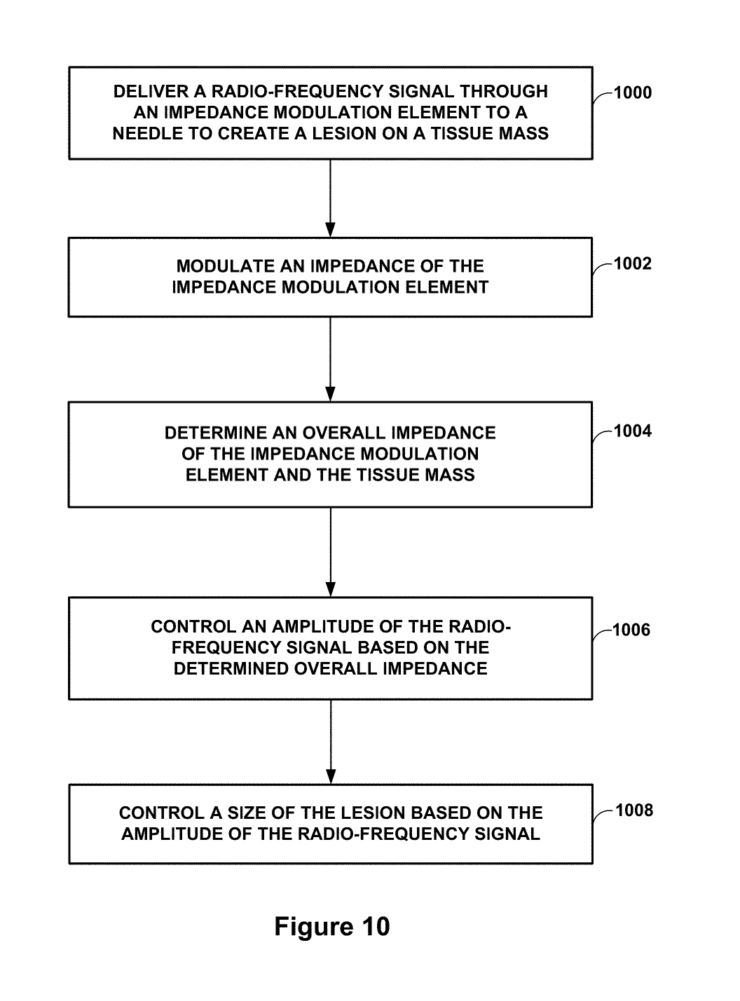

FIG. 10 is a flowchart illustrating an example operation in accordance with the techniques of the disclosure. The techniques of FIG. 10 are described with reference to processor 12 shown in FIG. 1B, although other components may exemplify similar techniques. FIG. 10 includes operations 1000 through 1008, which may be performed in a different order than what is shown in FIG. 10. Additional operations, beyond operations 1000 through 1008, may be performed in other examples. Processor 12 may be configured to perform any of operations 1000 through 1008, or any and all other techniques described with respect to FIG. 10.

In the example of FIG. 10, processor 12 causes impedance-modulation RF device 10 to deliver a radio-frequency signal through impedance modulation element 20 to RF probe 7 to create a lesion on body tissue 9 (1000). Impedance modulation element 20 may include a rheostat, a potentiometer, a variable resistor, and/or other circuit elements. Through impedance-modulation RF device 10, processor 12 can control the voltage, current, and power of the radio-frequency signal to body tissue 9. RF apparatus 5 includes a signal generator for producing an electrical signal and for controlling the electrical parameters of the electrical signal.

In the example of FIG. 10, processor 12 modulates an impedance of impedance modulation element 20 (1002). Processor 12 can modulate the impedance by connecting or disconnecting resistors in parallel with impedance modulation element 20. The modulated impedance and configuration (e.g., series, parallel, a combination thereof, etc.) of impedance modulation element 20 affects the electrical parameters of the RF signal that is delivered to body tissue 9. For example, by connecting impedance modulation element 20 in series with body tissue 9 can decrease the voltage drop across body tissue 9.

In the example of FIG. 10, processor 12 determines an overall impedance of impedance modulation element 20 and body tissue 9 (1004). Processor 12 can receive a signal from a sensor, where the signal indicates the overall impedance across impedance modulation element 20 and body tissue 9. Processor 12 can determine the overall impedance by determining the impedance between RF probe 7 and the grounding electrode. The overall impedance also includes the impedance across impedance modulation element 20 in examples in which impedance modulation element 20 is connected in series with body tissue 9. In some examples, the sensor is part of RF probe 7 or part of the grounding electrode.

In the example of FIG. 10, processor 12 controls an amplitude of the radio-frequency signal based on the determined overall impedance (1006). Processor 12 can control the amplitude of the radio-frequency signal by modulating the impedance of impedance modulation element 20. For example, processor 12 can connect or disconnect resistors of impedance modulation element 20 to increase or decrease the impedance across impedance modulation element 20. Additionally or alternatively, processor 12 can control the amplitude of the radio-frequency signal by modulating the current, voltage, or power of the radio-frequency signal. Processor 12 can cause RF apparatus 5 to increase or decrease the amplitude by increasing or decreasing the voltage or current of the signal delivered by RF apparatus 5 to impedance-modulation RF device 10.

In the example of FIG. 10, processor 12 controls a size of the lesion based on the amplitude of the radio-frequency signal (1008). The size of the lesion created by the radio-frequency signal is based on several factors such as the power dissipated in body tissue 9, the water content of body tissue 9, and the temperature of body tissue 9. By modulating the impedance of impedance modulation element 20, processor 12 can have more control over the size of the lesion in body tissue 9, as compared to another device that only controls the current or voltage of the RF signal.

The following numbered examples demonstrate one or more aspects of the disclosure.

### EXAMPLE 1

A method for ablating a tissue mass, the method including delivering a radio-frequency signal through an impedance modulation element to an introducer (e.g., with a conductive active tip) to create a lesion on the tissue mass. The method also includes modulating an impedance of the impedance modulation element, determining an overall impedance of the impedance modulation element and the tissue mass, and controlling an amplitude of the radio-frequency signal based on the determined overall impedance. The method further includes controlling a size of the lesion based on the amplitude of the radio-frequency signal.

### EXAMPLE 2

The method of example 1, where modulating the impedance of the impedance modulation element includes modulating the impedance of the impedance modulation element based on a defined size of the lesion.

### EXAMPLE 3

The method of any combination of example 1 or 2, further including receiving a user input.

### EXAMPLE 4

The method of any combination of examples 1-3, further including receiving a user input for selecting a size of the needle.

### EXAMPLE 5

The method of any combination of examples 1-4, further including receiving a user input for selecting a location of the tissue mass.

### EXAMPLE 6

The method of any combination of examples 1-5, further including receiving a user input for selecting a target amplitude.

### EXAMPLE 7

The method of any combination of examples 1-6, further including determining the defined size of the lesion based on the user input.

### EXAMPLE 8

The method of any combination of examples 1-7, where the user input indicates the defined size of the lesion and a size of the needle.

### EXAMPLE 9

The method of any combination of examples 1-8, further including determining a target amplitude of the radio-frequency signal based on the defined size of the lesion.

### EXAMPLE 10

The method of any combination of examples 1-9, further including determining a target amplitude of the radio-frequency signal based on the size of the introducer.

### EXAMPLE 11

The method of any combination of examples 1-10, further including determining a target amplitude of the radio-frequency signal based on the determined overall impedance.

### EXAMPLE 12

The method of any combination of examples 1-11, further including determining that the defined size of the lesion is not within an acceptable range of sizes.

### EXAMPLE 13

The method of any combination of examples 1-12, further including outputting a suggested size of the lesion to the user in response to determining that the defined size of the lesion is not within the acceptable range of sizes.

### EXAMPLE 14

The method of any combination of examples 1-13, further including determining that the defined size of the lesion is less than a threshold.

### EXAMPLE 15

The method of any combination of examples 1-14, further including connecting a resistance of the impedance modulation element in parallel with the tissue mass in response to determining that the defined size of the lesion is less than the threshold.

### EXAMPLE 16

The method of any combination of examples 1-15, further including switching between a first configuration where the impedance modulation element is connected in parallel with the tissue and a second configuration where the impedance modulation element is connected in series with the tissue.

### EXAMPLE 17

The method of any combination of examples 1-16, further including determining a power profile for ablating the tissue mass based on the defined size of the lesion.

### EXAMPLE 18

The method of any combination of examples 1-17, where modulating the impedance of the impedance modulation element is based on the power profile.

### EXAMPLE 19

The method of any combination of examples 1-18, further including modulating the impedance of the impedance modulation element based on the user input.

### EXAMPLE 20

The method of any combination of examples 1-19, further including determining a target size of the lesion based on the user input.

### EXAMPLE 21

The method of any combination of examples 1-20, where modulating the impedance of the impedance modulation element is based on the target size of the lesion.

### EXAMPLE 22

The method of any combination of examples 1-21, further including outputting one or more possible probe sizes to the user based on the location of the tissue mass.

### EXAMPLE 23

The method of any combination of examples 1-22, further including modulating the impedance of the impedance modulation element based on the target amplitude,

### EXAMPLE 24

The method of any combination of examples 1-23, where modulating the impedance of the impedance-modulation system includes decreasing the impedance of the impedance-modulation system to increase the amplitude of the radio-frequency signal.

### EXAMPLE 25

The method of any combination of examples 1-24, where decreasing the impedance of the impedance-modulation system includes creating a surge in the amplitude of the radio-frequency signal.

### EXAMPLE 26

The method of any combination of examples 1-25, further including reducing a current of the radio-frequency signal after decreasing the impedance of the impedance-modulation system.

### EXAMPLE 27

The method of any combination of examples 1-26, further including reducing a current of the radio-frequency signal to maintain a temperature of the tissue mass below a threshold level.

### EXAMPLE 28

The method of any combination of examples 1-27, further including receiving a sensed signal indicating a temperature of the tissue mass; and

### EXAMPLE 29

The method of any combination of examples 1-28, further including controlling, using a control loop based on the sensed signal, a current of the radio-frequency signal,

### EXAMPLE 30

The method of any combination of examples 1-29, where the impedance modulation element includes a rheostat or a potentiometer.

### EXAMPLE 31

A system for ablating a tissue mass, where the system includes an introducer needle or cannula with a conductive active tip, an impedance modulation element, a signal generator configured to deliver a radio-frequency signal through the impedance modulation element to the introducer to create a lesion on the tissue mass. The system also includes processing circuitry configured to modulate an impedance of the impedance modulation element and determine an overall impedance of the impedance modulation element and tissue mass. The processing circuitry is further configured to control an amplitude of the radio-frequency signal based on the determined overall impedance and control a size of the lesion based on the amplitude of the radio-frequency signal.

### EXAMPLE 32

The system of example 31, further including a probe for sensing the overall impedance of the impedance modulation element and tissue mass.

### EXAMPLE 33

The system of any combination of example 31 or 32, where the impedance modulation element includes a rheostat or a potentiometer.

### EXAMPLE 34

The system of any combination of examples 31-33, where the processing circuitry is configured to perform the method of examples 1-30 or any combination thereof.

### EXAMPLE 35

A device includes a computer-readable medium having executable instructions stored thereon, configured to be executable by processing circuitry for causing the processing circuitry to cause a signal generator to deliver a radio-frequency signal through an impedance modulation element to an introducer to create a lesion on a tissue mass. The instructions are further configured to cause the processing circuitry to modulate an impedance of the impedance modulation element and determine an overall impedance of the impedance modulation element and the tissue mass. The instructions are further configured to control an amplitude of the radio-frequency signal based on the determined overall impedance and control a size of the lesion based on the amplitude of the radio-frequency signal.

### EXAMPLE 36

The device of example 35, where the instructions are configured to cause the processing circuitry to perform the method of examples 1-30 or any combination thereof.

It will be appreciated that any of the aspects, features and options described in view of the methods apply equally to the system and devices described herein. It will be understood that any one or more of the above aspects, features and options as described herein can be combined,

It will be obvious to those recently skilled in the art that modifications to the apparatus and process disclosed herein may occur, including substitution of various component values or nodes of connection, without parting from the true spirit and scope of the disclosure. For example, the disclosed apparatus and techniques can be extended for use in other procedures similar or different than those described herein.

The techniques described in this disclosure may be implemented, at least in part, in hardware, software, firmware or any combination thereof. For example, various aspects of the described techniques, such as impedance-modulation RF device 10 and/or processor 12, may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure, such as impedance-modulation RF device 10 and/or processor 12, may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A method for ablating a tissue mass, the method comprising:
delivering a radio-frequency signal through an impedance modulation element to an introducer to create a lesion on the tissue mass;
modulating an impedance of the impedance modulation element;
determining an overall impedance of the impedance modulation element and the tissue mass;
controlling an amplitude of the radio-frequency signal based on the determined overall impedance; and
controlling a size of the lesion based on the amplitude of the radio-frequency signal.

2. The method of claim 1, wherein modulating the impedance of the impedance modulation element comprises modulating the impedance of the impedance modulation element based on a defined size of the lesion.

3. The method of claim 2, further comprising:
receiving a user input; and
determining the defined size of the lesion based on the user input.

4. The method of claim 3,
wherein the user input indicates the defined size of the lesion and a size of the introducer, and
wherein the method further comprises determining a target amplitude of the radio-frequency signal based on the defined size of the lesion, the size of the introducer, and the determined overall impedance.

5. The method of claim 3, further comprising:
determining that the defined size of the lesion is not within an acceptable range of sizes; and
outputting a suggested size of the lesion to the user in response to determining that the defined size of the lesion is not within the acceptable range of sizes.

6. The method of claim 2, further comprising:
determining that the defined size of the lesion is less than a threshold; and
connecting a resistance of the impedance modulation element in parallel with the tissue mass in response to determining that the defined size of the lesion is less than the threshold.

7. The method of claim 1, further comprising switching between:
a first configuration where the impedance modulation element is connected in parallel with the tissue: and
a second configuration where the impedance modulation element is connected in series with the tissue.

8. The method of claim 2, further comprising determining a power profile for ablating the tissue mass based on the defined size of the lesion,
wherein modulating the impedance of the impedance modulation element is based on the power profile.

9. The method of claim 1, further comprising:
receiving a user input for selecting a size of the introducer; and
modulating the impedance of the impedance modulation element based on the user input.

10. The method of claim 1, further comprising:
receiving a user input for selecting a location of the tissue mass; and
modulating the impedance of the impedance modulation element based on the user in

11. The method of claim 10, further comprising determining a target size of the lesion based on the user input,
wherein modulating the impedance of the impedance modulation element is based on the target size of the lesion.

12. The method of claim 10, further comprising outputting one or more possible probe and introducer sizes to the user based on the location of the tissue mass.

13. The method of claim 1, further comprising:
receiving a user input for selecting a target amplitude; and
modulating the impedance of the impedance modulation element based on the target amplitude.

14. The method of claim 13, wherein decreasing the impedance of the impedance-modulation system comprises creating a surge in the amplitude of the radio-frequency signal.

15. A system for ablating a tissue mass, the system comprising:
an introducer;
an impedance modulation element;
a signal generator configured to deliver a radio-frequency signal through the impedance modulation element to the introducer to create a lesion on the tissue mass; and
processing circuitry configured to:
modulate an impedance of the impedance modulation element;
determine an overall impedance of the impedance modulation element and tissue mass;
control an amplitude of the radio-frequency signal based on the determined overall impedance;
control a size of the lesion based on the amplitude of the radio-frequency signal.

16. The system of claim 15, further comprising an input device configured to receive a user input,
wherein the processor is configured to modulate the impedance of the impedance modulation element by modulating the impedance of the impedance modulation element based on a defined size of the lesion, and
wherein the processor is further configured to determine the defined size of the lesion based on the user input.

17. The system of claim 15, further comprising an input device configured to receive a user input, wherein the processor is configured to:
determine that the defined size of the lesion is less than a threshold; and
connect a resistance of the impedance modulation element in parallel with the tissue mass in response to determining that the defined size of the lesion is less than the threshold.

18. The system of claim 15, wherein the impedance modulation element comprises a rheostat or a potentiometer.

19. The system of claim 15, further comprising a probe for sensing the overall impedance of the impedance modulation element and tissue mass.
